(19) United States
(12) Patent Application Publication
(10) Pub. No.: US 2019/0132655 A1
(43) Pub. Date: May 2, 2019

(54) SENSOR MODULE, CONNECTOR MODULE, AND WATER QUALITY MEASURING DEVICE COMPRISING THE SAME

(71) Applicant: Dae Yoon Scale Industrial Co., Ltd., Seoul (KR)

(72) Inventors: In Ho SEO, Gyeonggi-do (KR); Youn Gyu CHOI, Seoul (KR); In Jun YOON, Seoul (KR); Guoo Sung PARK, Gyeonggi-do (KR); Eun Jung JI, Seoul (KR); Kwon Seok KIM, Seoul (KR)

(21) Appl. No.: 16/080,733

(22) PCT Filed: Mar. 10, 2017
(86) PCT No.: PCT/KR2017/002624

§ 371 Date: Aug. 29, 2018

(30) Foreign Application Priority Data
Mar. 11, 2016 (KR) 10-2016-0029305

(51) Int. Cl. H04Q 9/00; G01N 33/18

(52) U.S. Cl. CPC H04Q 9/00; H02J 50/10; G01N 33/18

(57) ABSTRACT

A sensor module according to an embodiment of the present invention is connected to a connector module, which transfers water quality information to an electronic device such that the same is displayed on the electronic device, and thereby acquires water quality characteristics for calculating the information. The sensor module is separably connected to the connector module and can be connected to any connector module. The connector module comprises an electronic device communication portion for transferring the water quality information to the electronic device, a body-proximate communication portion capable of wirelessly communicating with the sensor module, and a wireless power transmitting portion for wirelessly supplying the sensor module with power. The sensor module comprises: a sensor portion for acquiring water quality data for calculating the water quality information; a sensor-proximate communication portion for wirelessly transmitting the water quality data, which has been acquired by the sensor portion, to the body-proximate communication portion; a wireless power receiving portion for wirelessly receiving the power from the wireless power transmitting portion; and a sensor body portion on which the sensor-proximate communication portion and the wireless power receiving portion are mounted. The sensor body portion is connected to the connector module such that the sensor-proximate communication portion and the wireless power receiving portion are fixed in predetermined positions on the connector module. The sensor-proximate communication portion and the wireless power receiving portion may be positioned and spaced from the body-proximate communication portion and the wireless power transmitting portion such that, when the sensor body portion and the connector module are connected to each other, an abnormally excessive current is prevented from flowing to at least one of the body-proximate communication portion and the wireless power transmitting portion.

### TECHNICAL FIELD

The present invention relates to a sensor module for acquiring water-quality information by measuring water quality, a connector module, and a water quality measuring device including the same.

### BACKGROUND ART

A water quality measuring device may mean a device for measuring several measurement items of sewage, waste water, service water, lake water, cooling water or washing water used in a process of electric power generation, a general production line, or the like with one measuring instrument and for transmitting resultant data.

When necessary, a water quality measuring device performs hydrogen ion measurement, dissolved oxygen measurement, electric conductivity measurement, biochemical oxygen demand (BOD) measurement, floating particle measurement, total nitrogen measurement, total phosphorus measurement, chloride ion measurement, etc. which are used in the fields of water quality control and water-quality measurement monitoring and control.

Such an existing water quality measuring device is individually configured for each measurement item and used to measure water quality.

However, to measure various components of water quality and characteristics of several items, a water quality measuring device suited to each measurement item is individually used for measurement such that measurement is complicated, and purchasing and running multiple water quality measuring devices entail high cost.

In addition, an existing water quality measuring device is configured as one device or system which acquires water quality data by using a sensor, convert the acquired data into water quality information through a control portion, and display the converted information to let a user recognize the information.

In this case, when there is an issue in the water quality measuring device, a user cannot know which one among a part which acquires the water quality data by using the sensor, a part which converts the acquired data into the water quality information, and a part which displays the converted information such that the converted information may be recognized by the user is malfunctioning.

Further, since the respective parts are connected as one device, an abnormal overcurrent generated at any one part has influence on other parts, and cause all the parts to malfunction as a result.

### Technical Problem

The present invention is directed to implementing individual water-quality measuring sensor modules capable of measuring hydrogen ions, dissolved oxygen, electric conductivity, biochemical oxygen demand (BOD), floating particles, total nitrogen, total phosphorus, chloride ions, etc., and a connector module which may be connected to an arbitrary one of the plurality of sensor modules and thus acquires water quality information from the arbitrary sensor module and transfer the information to an electronic device capable of displaying the information to a user, such that the user need not purchase individual water quality measuring devices capable of measuring hydrogen ions, dissolved oxygen, electric conductivity, BOD, floating particles, etc. and may acquire various kinds of water quality information by only purchasing individual water-quality measuring sensor modules capable of measuring hydrogen ions, dissolved oxygen, electric conductivity, BOD, floating particles, etc.

The present invention is also directed to individually configuring a water-quality measuring module and a connector module so that a user can easily decide which part is malfunctioning and a failure of any one part has no influence on other parts.

### Technical Solution

One aspect of the present invention provides a sensor module which is connected to a connector module for transferring water quality information to an electronic device so that the electronic device displays the water quality information, and acquires water quality characteristics for calculating the water quality information, the connector module including an electronic device communication portion for transferring the water quality information to the electronic device, a body-proximate communication portion capable of wirelessly communicating with the sensor module, and a wireless power transmitting portion for wirelessly supplying power to the sensor module, and the sensor module being detachably connected to the connector module and thus able to be connected to an arbitrary connector module and including: a sensor portion configured to acquire water quality data for calculating the water quality information; a sensor-proximate communication portion configured to wirelessly transmit the water quality data acquired by the sensor portion to the body-proximate communication portion; a wireless power receiving portion configured to wirelessly receive the power from the wireless power transmitting portion; and a sensor body portion in which the sensor-proximate communication portion and the wireless power receiving portion are installed. The sensor body portion is connected to the connector module so that the sensor-proximate communication portion and the wireless power receiving portion are fixed at predetermined positions in the connector module, and when the sensor body portion and the connector module are connected, the sensor-proximate communication portion and the wireless power receiving portion are positioned to be spaced apart from the body-proximate communication portion and the wireless power transmitting portion so that an abnormal overcurrent is prevented from flowing to at least one of the body-proximate communication portion and the wireless power transmitting portion.

The sensor body portion of the sensor module according to an embodiment of the present invention may include: a body-connected body portion configured to provide a region to which the connector module is connected; a sensor-connected body portion configured to provide a region to which the sensor portion is connected; and a sensor-disposed body portion configured to make the sensor-proximate communication portion and the wireless power receiving portion be positioned at the predetermined positions spaced apart from the body-proximate communication portion and the wireless power transmitting portion.

The sensor-disposed body portion and the sensor-connected body portion of the sensor module according to an embodiment of the present invention may surround the sensor-proximate communication portion and the wireless power receiving portion so that external moisture is prevented from entering into the sensor-proximate communication portion and the wireless power receiving portion.

The sensor-disposed body portion of the sensor module according to an embodiment of the present invention may be attached to or detached from the sensor-connected body portion.

The sensor-proximate communication portion and the wireless power receiving portion of the sensor module according to an embodiment of the present invention may be positioned in the sensor-disposed body portion to be spaced apart from each other in a lengthwise direction of the sensor portion.

The sensor-disposed body portion of the sensor module according to an embodiment of the present invention may make the sensor-proximate communication portion and the wireless power receiving portion be disposed at the predetermined positions so that the body-proximate communication portion and the wireless power transmitting portion of the connector module are positioned to be spaced apart from the sensor-proximate communication portion and the wireless power receiving portion in a direction perpendicular to the lengthwise direction when the sensor module is connected to the connector module by the body-connected body portion.

Another aspect of the present invention provides a connector module which receives water quality information from a sensor module including a sensor portion, a sensor-proximate communication portion, and a wireless power receiving portion and transfers the water quality information to an electronic device to display the water quality information on the electronic device, the connector module including: a body-proximate communication portion configured to wirelessly receive the water quality information from the sensor-proximate communication unit of the sensor module; a wireless power transmitting portion configured to wirelessly supply power to the wireless power receiving portion of the sensor module; an electronic device communication portion configured to transfer the water quality information to the electronic device; and a main body portion in which the sensor-proximate communication portion and the wireless power receiving portion are installed. The main body portion is connected to the sensor module and thereby makes the body-proximate communication portion and the wireless power transmitting portion be fixed at predetermined positions in the sensor module, and when the main body portion and the sensor module are connected, the body-proximate communication portion and the wireless power transmitting portion are positioned to be spaced apart from the sensor-proximate communication portion and the wireless power receiving portion so that an abnormal overcurrent is prevented from flowing to at least one of the sensor-proximate communication portion and the wireless power receiving portion.

The main body portion of the sensor module according to an embodiment of the present invention may include: a device-connected body portion configured to be connected to the sensor module; and a body-disposed body portion configured to make the body-proximate communication portion and the wireless power transmitting portion be positioned at the predetermined positions spaced apart from the sensor-proximate communication portion and the wireless power receiving portion.

The main body portion of the sensor module according to an embodiment of the present invention may further include a communication-connecting body portion configured to provide a region to which the electronic device communication portion is connected, and the body-disposed body portion and the communication-connecting body portion may surround the body-proximate communication portion and the wireless power transmitting portion so that external moisture is prevented from entering into the body-proximate communication portion and the wireless power transmitting portion.

The body-disposed body portion of the sensor module according to an embodiment of the present invention may be attached to or detached from the communication-connecting body portion.

The body-proximate communication portion and the wireless power transmitting portion of the sensor module according to an embodiment of the present invention may be positioned in the body-disposed body portion to be spaced apart from each other in a lengthwise direction of the sensor portion.

The body-disposed body portion of the sensor module according to an embodiment of the present invention may make the body-proximate communication portion and the wireless power transmitting portion be disposed at the predetermined positions so that the sensor-proximate communication portion and the wireless power receiving portion of the sensor module are positioned to be spaced a predetermined distance from the body-proximate communication portion and the wireless power transmitting portion in a direction perpendicular to the lengthwise direction when the connector module is connected to the sensor module by the device-connected body portion.

The body-connected body portion of the sensor module according to an embodiment of the present invention may include: a first device-connected body portion configured to be connected to the body-disposed body portion; and a second device-connected body portion configured to connect the first body-connected body portion and the sensor module.

The first device-connected body portion of the sensor module according to an embodiment of the present invention may surround the sensor-disposed body portion and the body-disposed body portion when the connector module is connected to the sensor module.

The main body portion of the sensor module according to an embodiment of the present invention may further include a communication-connecting body portion configured to provide a region to which the electronic device communication portion is connected, and the body-disposed body portion may include: a first body-disposed body portion configured to make the body-proximate communication portion and the wireless power transmitting portion be disposed at the predetermined positions; a second body-disposed body portion configured to provide a region to which the communication-connecting body portion is connected; a third body-disposed body portion configured to provide a region to which the device-connected body portion is connected; and a fourth body-disposed body portion configured to define a boundary between the second body-disposed body portion and the third body-disposed body portion.

Another aspect of the present invention provides a water quality measuring device including the sensor module and the connector module.

### Advantageous Effects

A sensor module, a connector module, and a water quality measuring device including the same according to an embodiment of the present invention make it possible to acquire various kinds of water quality information by using several sensor modules through one connector module, and thus can be conveniently and economically used by a user.

Further, a connector module and a sensor module are independently configured. Therefore, a user can easily determine a malfunctioning part when there are issues, other parts are not affected when an abnormal overcurrent is generated, such that utilization can be maximized.

### DESCRIPTION OF DRAWINGS

FIG. 1 is a schematic perspective view of a water quality measuring device according to an embodiment of the present invention.

FIG. 2 is a schematic exploded perspective view of the water quality measuring device according to the embodiment of the present invention.

FIG. 3 is a schematic perspective view of a sensor module according to the embodiment of the present invention.

FIG. 4 is a schematic exploded perspective view of the sensor module according to the embodiment of the present invention.

FIG. 5 is a schematic perspective view of a connector module according to the embodiment of the present invention.

FIG. 6 is a schematic exploded perspective view of the connector module according to the embodiment of the present invention.

FIG. 7 is a schematic cross-sectional view of the water quality measuring device according to an embodiment of the present invention.

FIG. 8 is a schematic exploded perspective view of the connector module according to an embodiment of the present invention.

FIG. 9 is a schematic exploded perspective view of the water quality measuring device according to the embodiment of the present invention.

### MODES OF THE INVENTION

Hereinafter, embodiments of the present invention will be described in detail with reference to the accompanying drawings. However, the spirit of the present invention is not limited to the suggested embodiments, and those of ordinary skill in the art may easily suggest another retrogressive invention or another embodiment, which falls within the spirit of the present invention, through addition, modification, deletion, etc. of a component without departing from the spirit of the present invention.

Elements which are shown in the drawings of the respective embodiments and have the same function within the same spirit will be described with like reference signs.

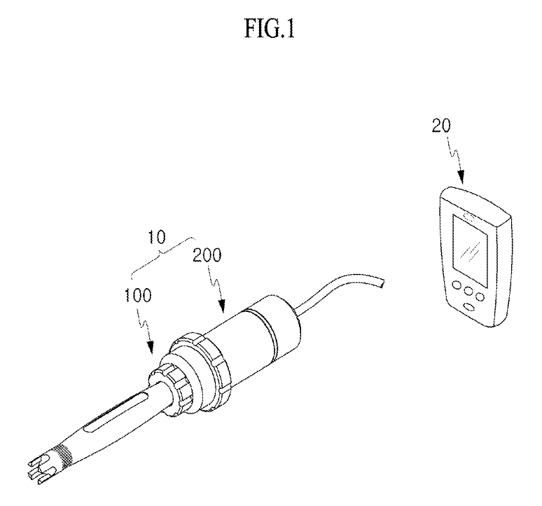

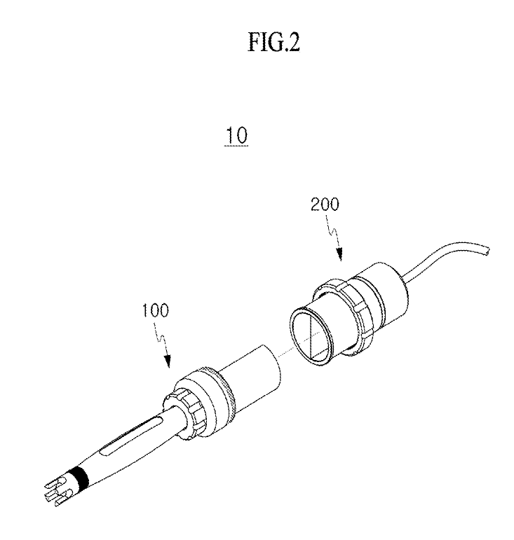

FIG. 1 is a schematic perspective view of a water quality measuring device according to an embodiment of the present invention, and FIG. 2 is a schematic exploded perspective view of the water quality measuring device according to the embodiment of the present invention.

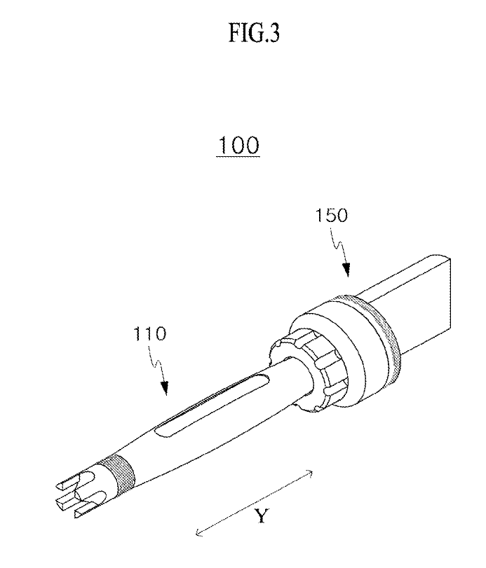

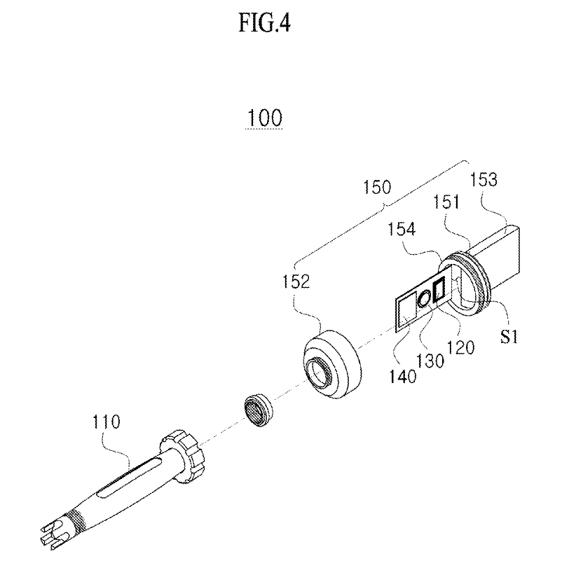

FIG. 3 is a schematic perspective view of a sensor module according to the embodiment of the present invention, and FIG. 4 is a schematic exploded perspective view of the sensor module according to the embodiment of the present invention.

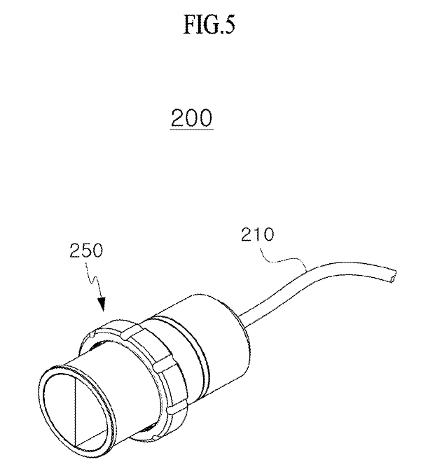

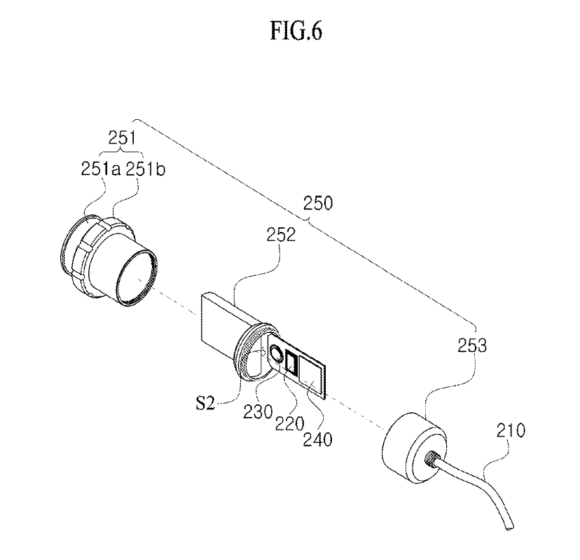

FIG. 5 is a schematic perspective view of a connector module according to the embodiment of the present invention, and FIG. 6 is a schematic exploded perspective view of the connector module according to the embodiment of the present invention.

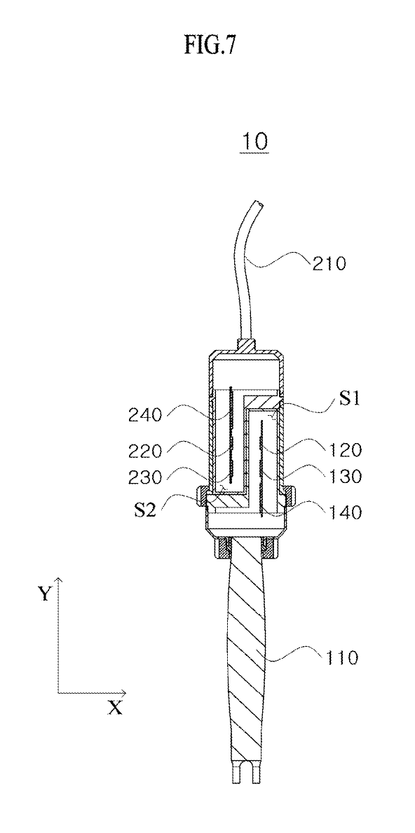

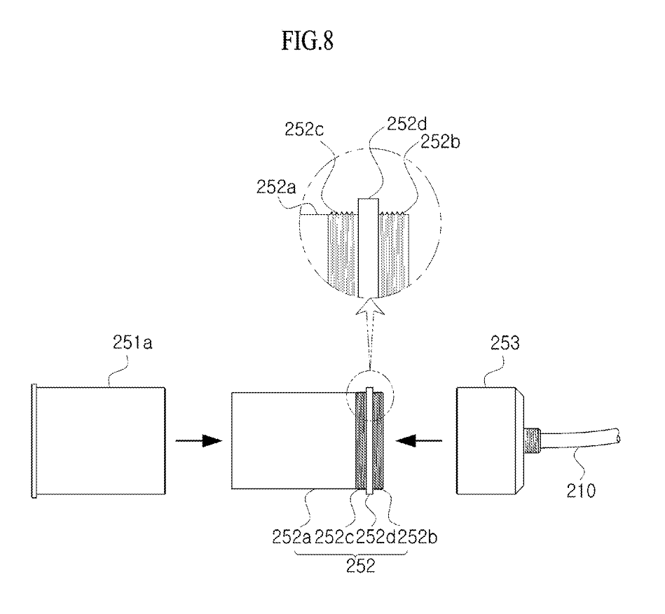

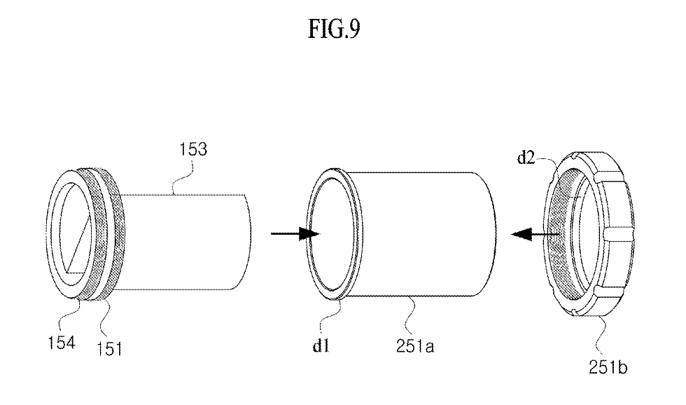

FIG. 7 is a schematic cross-sectional view of the water quality measuring device according to the embodiment of the present invention, FIG. 8 is a schematic exploded perspective view of a connector module according to the embodiment of the present invention, and FIG. 9 is a schematic exploded perspective view of the water quality measuring device according to the embodiment of the present invention.

FIGS. 1 to 9 are diagrams schematically showing a configuration of a water quality measuring device 10 according to the embodiment of the present invention. For example, interconnections between components, circuit diagrams, etc. which are not core elements of the technical spirit of the present invention are omitted to clearly describe the present invention. Therefore, although elements, such as interconnections, circuit diagrams, etc., are omitted in FIGS. 1 to 9, it is possible to implement functions of the present invention.

First, terms related to directions will be defined. A lengthwise direction Y may denote a vertical direction as shown in FIG. 7, and a direction X perpendicular to the lengthwise direction Y may denote a lateral direction.

As shown in FIGS. 1 to 9, a sensor module 100 according to the embodiment of the present invention is a device which is connected to a connector module 200 for displaying water quality information on an electronic device 20 and acquires water quality data for calculating the water quality information. The sensor module 100 may be detachably connected to the connector module 200 and may thus be connected to an arbitrary connector module 200. The connector module 200 includes an electronic device communication portion 210 for transferring the water quality data to the electronic device 20, a body-proximate communication portion 220 capable of wirelessly communicating with the sensor module 100, and a wireless power transmitting portion 230 for wirelessly supplying power to the sensor module 100. The sensor module 100 may include a sensor portion 110 for acquiring the water quality data for calculating the water quality information, a sensor-proximate communication portion 120 for wirelessly transmitting the water quality data acquired by the sensor portion 110 to the body-proximate communication portion 220, a wireless power receiving portion 130 for wirelessly receiving the power from the wireless power transmitting portion 230, and a sensor body portion 150 in which the sensor-proximate communication portion 120 and the wireless power receiving portion 130 are installed.

Here, the water quality information is information which enables a user to decide water quality characteristics, and may be a value, a graph, an image, a sound, and the like.

The water quality data is data acquired by the sensor portion 110 or data calculated or converted from the acquired water quality data by a control portion or a calculation portion. The water quality information may be generated on the basis of the water quality data.

In other words, the water quality data may denote all data with which it is possible to calculate a value, a graph, an image, a sound, etc., which are the water quality information displayed on the electronic device 20.

The electronic device 20 is a device capable of displaying the water quality information to a user, and may be, for example, a portable terminal, electronic equipment, a smart phone, and the like.

Although described in further detail below, the sensor module 100 may be a device which acquires the water quality data through the sensor portion 110, and the connector module 200 may be a device which receives the water quality data from the sensor module 100 and transfers the water quality data to the electronic device 20.

Here, the sensor module 100 may be a device which acquires water quality data for measuring hydrogen ions, dissolved oxygen, electric conductivity, biochemical oxygen demand (BOD), floating particles, total nitrogen, total phosphorus, chloride ions, etc., according to functions of the sensor portion 110, and respective sensor modules 100 may be independently implemented according to the functions.

For example, there may be a sensor module 100 for measuring hydrogen ions, and there may be a sensor module 100 for measuring dissolved oxygen separately from the sensor module 100.

Also, a sensor module 100 may have a plurality of functions among several functions.

Here, the connector module 200 may be attached to or detached from an arbitrary one of the plurality of sensor modules 100. When the connector module 200 is connected to any one of the sensor modules 100, the connector module 200 may acquire the water quality data from the connected sensor module 100.

In other words, the connector module 200 may be compatibly connected to several sensor modules 100 and may perform a function thereof.

The sensor portion 110 may acquire the water quality data by coming into direct contact with water whose quality will be analyzed or without coming into contact with the water.

For example, the sensor portions 110 may be a hydrogen ion sensor, a dissolved oxygen sensor, an electric conductivity sensor, a floating particle sensor, a chloride ion sensor, etc., according to kinds of water quality data to be acquired.

The respective sensor portions 110 may vary in shape, size, and material according to functions thereof.

The sensor-proximate communication portion 120 may wirelessly transmit the water quality data acquired by the sensor portions 110 to the body-proximate communication portion 220 of the connector module 200.

The sensor-proximate communication portion 120 and the body-proximate communication portion 220 may denote short-range communication modules.

For example, the sensor-proximate communication portion 120 and the body-proximate communication portion 220 may be Bluetooth modules, radio frequency identification (RFID) modules, infrared data association (IrDA) modules, ultra wideband (UWB) modules, ZigBee modules, wireless high-definition (WiHD) modules, WiGig modules, near field communication (NFC) modules, or the like.

In other words, the sensor-proximate communication portion 120 and the body-proximate communication portion 220 may denote any component capable of wireless communication.

The wireless power receiving portion 130 may be a component which wirelessly receives power from the wireless power transmitting portion 230 of the sensor body device.

For example, wireless power transmission and reception between the wireless power receiving portion 130 and the wireless power transmitting portion 230 may be performed in a magnetic induction manner, a magnetic resonance manner, or the like.

In other words, the wireless power receiving portion 130 and the wireless power transmitting portion 230 may denote any component capable of wirelessly transmitting and receiving power.

The sensor modules 100 according to the embodiment of the present invention may further include a sensor control portion 140.

The sensor control portion 140 may store and process the water quality data acquired from the sensor portions 110, and control the sensor-proximate communication portion 120 and the wireless power receiving portion 130.

The sensor body portion 150 may be connected to the connector module 200 and thereby make the sensor-proximate communication portion 120 and the wireless power receiving portion 130 be fixed at predetermined positions in the connector module 200. More specifically, when the sensor modules 100 are connected to the connector module 200, the sensor body portion 150 may allow the sensor-proximate communication portion 120 and the wireless power receiving portion 130 to be positioned at the predetermined positions from the connector module 200.

In other words, the sensor body portion 150 provides a region in which the sensor-proximate communication portion 120 and the wireless power receiving portion 130 may be positioned at the predetermined positions. For example, the sensor-proximate communication portion 120 and the wireless power receiving portion 130 may be connected, installed, or fixed on the sensor body portion 150 and thereby positioned at the predetermined positions.

Therefore, when the sensor modules 100 are connected to the connector module 200, the sensor-proximate communication portion 120 and the wireless power receiving portion 130 may be fixed at the predetermined positions from the connector module 200.

Here, as shown in FIG. 7, when the sensor body portion 150 and the connector module 200 are connected, the sensor-proximate communication portion 120 and the wireless power receiving portion 130 may be positioned to be spaced apart from the body-proximate communication portion 220 and the wireless power transmitting portion 230 so that an abnormal overcurrent is prevented from flowing to at least one of the body-proximate communication portion 220 and the wireless power transmitting portion 230.

The sensor portions 110 may come into direct contact with the water whose quality will be measured to acquire the water quality data, and an abnormal overcurrent may flow to the sensor-proximate communication portion 120 and the wireless power receiving portion 130 due to the sensor portions 110 immersed into the water.

Here, since the sensor-proximate communication portion 120 and the wireless power receiving portion 130 are positioned to be spaced apart from the body-proximate communication portion 220 and the wireless power transmitting portion 230, respectively, the abnormal overcurrent flowing through the sensor-proximate communication portion 120 and the wireless power receiving portion 130 is prevented from flowing to the body-proximate communication portion 220 and the wireless power transmitting portion 230. As a result, it is possible to prevent malfunction of the connector module 200 caused by the abnormal overcurrent.

Also, since the sensor-proximate communication portion 120 and the wireless power receiving portion 130 do not come into direct contact with the body-proximate communication portion 220 and the wireless power transmitting portion 230, respectively, a user need not connect the sensor-proximate communication portion 120 and the body-proximate communication portion 220 or the wireless power receiving portion 130 and the wireless power transmitting portion 230 when connecting the sensor modules 100 to the connector module 200.

Therefore, when any one of the plurality of sensor modules 100 is connected to the connector module 200, it is possible to efficiently replace the sensor module 100 with another sensor module 100, and the probability of malfunction lowers.

Here, FIG. 7 shows that the sensor-proximate communication portion 120 and the wireless power receiving portion 130 are spaced apart from the body-proximate communication portion 220 and the wireless power transmitting portion 230 in the direction X perpendicular to the lengthwise direction Y. However, positions of the sensor-proximate communication portion 120 and the wireless power receiving portion 130 are not limited thereto, and the sensor-proximate communication portion 120 and the wireless power receiving portion 130 may be positioned to be spaced apart from the body-proximate communication portion 220 and the wireless power transmitting portion 230 in the lengthwise direction Y.

In other words, as long as the sensor-proximate communication portion 120 and the wireless power receiving portion 130 are positioned to be spaced a predetermined distance from the body-proximate communication portion 220 and the wireless power transmitting portion 230, a direction of the distance may vary according to a situation of those of ordinary skill in the art.

As shown in FIG. 4, for example, the sensor body portion 150 may include a body-connected body portion 151 for providing a region to which the connector module 200 is connected, a sensor-connected body portion 152 for providing a region to which the sensor portions 110 are connected, and a sensor-disposed body portion 153 for making the sensor-proximate communication portion 120 and the wireless power receiving portion 130 be positioned at the predetermined positions spaced apart from the body-proximate communication portion 220 and the wireless power transmitting portion 230.

For example, the body-connected body portion 151 may have a screw thread, and may be threadedly engaged with the connector module 200 which will be described in further detail below.

However, the connection between the body-connected body portion 151 and the connector module 200 is not limited to the thread engagement, and may be made with various coupling structures such as groove coupling and the like.

In other words, the body-connected body portion 151 is required only to provide a region to which the connector module 200 is connected, and it is apparent to those of ordinary skill in the art that the coupling manner may be variously modified.

The sensor-connected body portion 152 may provide a region to which the sensor portions 110 is connected.

For example, the connection between the sensor-connected body portion 152 and the sensor portions 110 may be implemented by thread engagement. However, the connection is not limited to the thread engagement, and the sensor-connected body portion 152 and the sensor portions 110 may be integrally formed.

In other words, the sensor-connected body portion 152 is required only to provide a region in which the sensor portions 110 is connected to the sensor body portion 150, and it is apparent to those of ordinary skill in the art that the sensor-connected body portion 152 may be variously modified.

The sensor-disposed body portion 153 may provide a region in which the sensor-proximate communication portion 120 and the wireless power receiving portion 130 may be positioned at the predetermined positions so that the sensor-proximate communication portion 120 and the wireless power receiving portion 130 are positioned at the predetermined positions spaced apart from the body-proximate communication portion 220 and the wireless power transmitting portion 230.

For example, as shown in FIG. 4, the sensor-disposed body portion 153 may provide a first disposition space S1 in which the sensor-proximate communication portion 120 and the wireless power receiving portion 130 may be positioned at the predetermined positions, and the sensor-proximate communication portion 120 and the wireless power receiving portion 130 may be inserted into the first disposition space S1.

FIG. 4 shows that the sensor-disposed body portion 153 provides the first disposition space S1 which is closed in the direction X perpendicular to the lengthwise direction Y, but the first disposition space S1 is not limited thereto. Although not shown in the drawing, for example, a hole may be formed in the direction X perpendicular to the lengthwise direction Y so that the first disposition space S1 may communicate with the outside.

In other words, as long as the sensor-disposed body portion 153 has a configuration in which the sensor-proximate communication portion 120 and the wireless power receiving portion 130 are disposed at the predetermined positions spaced apart from the body-proximate communication portion 220 and the wireless power transmitting portion 230, the sensor-disposed body portion 153 may provide an open or closed first disposition space S1, and may be variously modified from the viewpoint of those of ordinary skill in the art.

As shown in FIGS. 3 and 4, for example, the sensor-disposed body portion 153 and the sensor-connected body portion 152 may surround the sensor-proximate communication portion 120 and the wireless power receiving portion 130 to prevent external moisture from entering into the sensor-proximate communication portion 120 and the wireless power receiving portion 130.

For example, the sensor-disposed body portion 153 may be attached to or detached from the sensor-connected body portion 152.

As an example, a body thread portion 154 formed in the sensor-disposed body portion 153 may be threadedly attached to or detached from the sensor-connected body portion 152.

However, the connection between the sensor-disposed body portion 153 and the sensor-connected body portion 152 is not limited to the thread engagement, and may be made with various coupling structures such as groove coupling and the like.

As shown in FIG. 4, for example, the sensor-proximate communication portion 120 and the wireless power receiving portion 130 may be disposed in the sensor-disposed body portion 153 to be spaced apart from each other in the lengthwise direction Y of the sensor portions 110.

As shown in FIGS. 4 and 7, for example, when the sensor modules 100 are connected to the connector module 200 by the body-connected body portion 151, the sensor-disposed body portion 153 may make the sensor-proximate communication portion 120 and the wireless power receiving portion 130 be positioned at the predetermined positions so that the body-proximate communication portion 220 and the wireless power transmitting portion 230 of the connector module 200 are positioned to be spaced apart from the sensor-proximate communication portion 120 and the wireless power receiving portion 130 in the direction X perpendicular to the lengthwise direction Y.

In other words, the sensor-disposed body portion 153 is implemented so that, when the sensor modules 100 are connected to the connector module 200, the sensor-proximate communication portion 120 and the body-proximate communication portion 220 are spaced apart from each other by a predetermined distance in the direction X perpendicular to the lengthwise direction Y at the same level in the lengthwise direction and the wireless power receiving portion 130 and the wireless power transmitting portion 230 are spaced apart from each other in the same manner. Therefore, when a user simply connects the sensor modules 100 to the connector module 200, the body-proximate communication portion 220 and the wireless power transmitting portion 230 of the connector module 200 may be positioned to be spaced apart from the sensor-proximate communication portion 120 and the wireless power receiving portion 130 in the direction X perpendicular to the lengthwise direction Y by the sensor-disposed body portion 153.

Therefore, when a user connects the sensor modules 100 and the connector module 200, no additional position adjustment is required to make the sensor-proximate communication portion 120 and the body-proximate communication portion 220 face each other and make the wireless power receiving portion 130 and the wireless power transmitting portion 230 face each other. As a result, it is possible to improve operational efficiency.

The connector module 200 to which the sensor modules 100 may be connected will be described in further detail below.

As shown in FIGS. 2, 5, and 6, the connector module 200 according to the embodiment of the present invention receives the water quality data from the sensor modules 100, which includes the sensor portions 110, the sensor-proximate communication portion 120, and the wireless power receiving portion 130, and transfers the water quality data to the electronic device 20 so that the water quality information is displayed on the electronic device 20. The connector module 200 may include the body-proximate communication portion 220 for wirelessly receiving the water quality data from the sensor-proximate communication portion of the sensor modules 100, the wireless power transmitting portion 230 for wirelessly supplying power to the wireless power receiving portion 130 of the sensor modules 100, the electronic device communication portion 210 for transferring the water quality data to the electronic device 20, and a main body portion 250 in which the sensor-proximate communication portion 120 and the wireless power receiving portion 130 are installed.

For example, the electronic device communication portion 210 is a component for allowing communication with the electronic device 20, and may communicate with the electronic device 20 in a wired manner or a wireless manner.

Therefore, the electronic device communication portion 210 may transfer the water quality data received from the sensor modules 100 to the electronic device 20.

For example, although not shown in the drawings, the connector module 200 may include a battery portion to supply the power from the wireless power transmitting portion 230 to the wireless power receiving portion 130, or may be supplied with external power in a wired manner.

For example, the connector module 200 may further include a body control portion 240 for controlling the body-proximate communication portion 220, the wireless power transmitting portion 230, and the electronic device communication portion 210, and the body control portion 240 may store, calculate, or process the water quality data received from the sensor modules 100 according to a selection.

The main body portion 250 may be connected to the sensor modules 100 and may thereby make the body-proximate communication portion 220 and the wireless power transmitting portion 230 be fixed at predetermined positions in the sensor modules 100.

More specifically, when the connector module 200 is connected to the sensor modules 100, the main body portion 250 may make the body-proximate communication portion 220 and the wireless power transmitting portion 230 be fixed at the predetermined positions from the sensor modules 100.

In other words, the main body portion 250 provides a region in which the body-proximate communication portion 220 and the wireless power transmitting portion 230 may be positioned at the predetermined positions. For example, the body-proximate communication portion 220 and the wireless power transmitting portion 230 may be connected, installed, or fixed on the main body portion 250 and thereby positioned at the predetermined positions.

Therefore, when the connector module 200 is connected to the sensor modules 100, the body-proximate communication portion 220 and the wireless power transmitting portion 230 may be fixed at the predetermined positions from the sensor module 100.

As shown in FIG. 7, when the main body portion 250 and the sensor modules 100 are connected, the body-proximate communication portion 220 and the wireless power transmitting portion 230 may be positioned to be spaced apart from the sensor-proximate communication portion 120 and the wireless power receiving portion 130 so that an abnormal overcurrent is prevented from flowing to at least one of the sensor-proximate communication portion 120 and the wireless power receiving portion 130.

FIG. 7 shows that the body-proximate communication portion 220 and the wireless power transmitting portion 230 are spaced apart from the sensor-proximate communication portion 120 and the wireless power receiving portion 130 in the direction X perpendicular to the lengthwise direction Y. However, the relative positions of the body-proximate communication portion 220 and the wireless power transmitting portion 230 are not limited thereto, and the body-proximate communication portion 220 and the wireless power transmitting portion 230 may be positioned to be spaced apart from the sensor-proximate communication portion 120 and the wireless power receiving portion 130 in the lengthwise direction Y.

In other words, as long as the body-proximate communication portion 220 and the wireless power transmitting portion 230 are positioned to be spaced a predetermined distance from the sensor-proximate communication portion 120 and the wireless power receiving portion 130, a direction of the distance may be variously modified from the viewpoint of those of ordinary skill in the art.

As shown in FIGS. 5, 6, and 9, the main body portion 250 may include a device-connected body portion 251 to which the sensor modules 100 are connected, and a body-disposed body portion 252 for making the body-proximate communication portion 220 and the wireless power transmitting portion 230 be disposed at the predetermined positions spaced apart from the sensor-proximate communication portion 120 and the wireless power receiving portion 130.

For example, the device-connected body portion 251 may be threadedly engaged with the body-connected body portion 151.

However, the connection between the device-connected body portion 251 and the body-connected body portion 151 is not limited to the thread engagement, and may be made with various coupling structures, such as groove coupling and the like.

The body-disposed body portion 252 may provide a region in which the body-proximate communication portion 220 and the wireless power transmitting portion 230 may be positioned at the predetermined positions so that the body-proximate communication portion 220 and the wireless power transmitting portion 230 are disposed at the predetermined positions spaced apart from the sensor-proximate communication portion 120 and the wireless power receiving portion 130.

For example, as shown in FIG. 6, the body-disposed body portion 252 may provide a second disposition space S2 in which the body-proximate communication portion 220 and the wireless power transmitting portion 230 may be positioned at the predetermined positions, and the body-proximate communication portion 220 and the wireless power transmitting portion 230 may be inserted into the second disposition space S2.

FIG. 6 shows that the body-disposed body portion 252 provides the second disposition space S2 which is closed in the direction X perpendicular to the lengthwise direction Y, but the second disposition space S2 is not limited thereto. Although not shown in the drawing, for example, a hole may be formed in the direction X perpendicular to the lengthwise direction Y so that the second disposition space S2 may communicate with the outside.

In other words, as long as the body-disposed body portion 252 has a configuration in which the body-proximate communication portion 220 and the wireless power transmitting portion 230 are disposed at the predetermined positions spaced apart from the sensor-proximate communication portion 120 and the wireless power receiving portion 130, the body-disposed body portion 252 may provide an open or closed second disposition space S2, and may be variously modified from the viewpoint of those of ordinary skill in the art.

As shown in FIG. 6, for example, the main body portion 250 may further include a communication-connecting body portion 253 for providing a region to which the electronic device communication portion 210 is connected, and the body-disposed body portion 252 and the communication-connecting body portion 253 may surround the body-proximate communication portion 220 and the wireless power transmitting portion 230 to prevent external moisture from entering into the body-proximate communication portion 220 and the wireless power transmitting portion 230.

The body-disposed body portion 252 may be attached to or detached from the communication-connecting body portion 253.

The body-disposed body portion 252 may be threadedly attached to or detached from the communication-connecting body portion 253. However, the attachment or detachment method is not limited to the thread engagement, and may be groove coupling and the like.

As shown in FIG. 7, for example, the body-proximate communication portion 220 and the wireless power transmitting portion 230 may be disposed in the body-disposed body portion 252 to be spaced apart from each other in the lengthwise direction Y.

When the connector module 200 is connected to the sensor modules 100 by the device-connected body portion 251, the body-disposed body portion 252 may make the body-proximate communication portion 220 and the wireless power transmitting portion 230 be disposed at the predetermined positions so that the sensor-proximate communication portion 120 and the wireless power receiving portion 130 of the sensor modules 100 may be positioned to be spaced apart from the body-proximate communication portion 220 and the wireless power transmitting portion 230 in the direction X perpendicular to the lengthwise direction Y.

In other words, the body-disposed body portion 252 is implemented so that, when the connector module 200 is connected to the sensor modules 100, the body-proximate communication portion 220 and the sensor-proximate communication portion 120 are spaced apart from each other by a predetermined distance in the direction X perpendicular to the lengthwise direction Y at the same level in the lengthwise direction and the wireless power transmitting portion 230 and the wireless power receiving portion 130 are spaced apart from each other in the same manner. Therefore, when a user simply connects the connector module 200 to the sensor modules 100, the sensor-proximate communication portion 120 and the wireless power receiving portion 130 of the sensor modules 100 may be positioned to be spaced apart from the body-proximate communication portion 220 and the wireless power transmitting portion 230 in the direction X perpendicular to the lengthwise direction Y by the body-disposed body portion 252.

For example, the body-disposed body portion 252 and the sensor-disposed body portion 153 may correspond to each other in shape and size.

As shown in FIGS. 6 and 9, the body-connected body portion 251 may include a first device-connected body portion 251a which is connected to the body-disposed body portion 252 and a second device-connected body portion 251b which connects the first device-connected body portion 251a and the sensor modules 100.

The first device-connected body portion 251a may be threadedly engaged with the body-disposed body portion 252. However, the connection between the first device-connected body portion 251a and the body-disposed body portion 251 is not limited to the thread engagement, and may be made with various coupling structures, such as groove coupling and the like.

For example, the second device-connected body portion 251b is implemented to connect the first device-connected body portion 251a and the sensor modules 100. When the second device-connected body portion 251b is rotated on and connected to the body thread portion 154, a protruding portion d2 of the second device-connected body portion 251b presses a protruding portion d1 of the first device-connected body portion 251a such that the first device-connected body portion 251a and the sensor modules 100 are connected.

As shown in FIGS. 2, 6, and 9, for example, when the connector module 200 is connected to the sensor modules 100, the first device-connected body portion 251a may surround the sensor-disposed body portion 153 and the body-disposed body portion 252.

For example, the first device-connected body portion 251a may have a cylindrical shape, and the sensor-disposed body portion 153 and the body-disposed body portion 252 may be inserted into the first device-connected body portion 251a.

As shown in FIGS. 6 and 8, for example, the body-disposed body portion 252 may include a first body-disposed body portion 252a for making the body-proximate communication portion 220 and the wireless power transmitting portion 230 be disposed at the predetermined positions, a second body-disposed body portion 252b for providing a region to which the communication-connecting body portion 253 is connected, a third body-disposed body portion 252c for providing a region to which the device-connected body portion 251 is connected, and a fourth body-disposed body portion 252d for defining a boundary between the second body-disposed body portion 252d and the third body-disposed body portion 252c.

The first body-disposed body portion 252a may provide the region for making the body-proximate communication portion 220 and the wireless power transmitting portion 230 be disposed at the predetermined positions.

The second body-disposed body portion 252b may provide the region to which the communication-connecting body portion 253 is connected, and may be, for example, threadedly engaged with the communication-connecting body portion 253.

However, the connection between the second body-disposed body portion 252b and the communication-connecting body portion 253 is not limited to the thread engagement, and may be implemented with groove coupling and the like.

The third body-disposed body portion 252c may provide the region to which the device-connected body portion 251 is connected, and may be, for example, threadedly engaged with the first device-connected body portion 251a of the device-connected body portion 251.

However, the connection between the third body-disposed body portion 252c and the device-connected body portion 251 is not limited to the thread engagement, and may be implemented with groove coupling and the like.

The fourth body-disposed body portion 252d may define the boundary between the second body-disposed body portion 252b and the third body-disposed body portion 252c. For example, the fourth body-disposed body portion 252d may be formed to protrude between the second body-disposed body portion 252b and the third body-disposed body portion 252c and disconnect the second body-disposed body portion 252b and the third body-disposed body portion 252c from each other.

The water quality measuring device 10 according to the embodiment of the present invention may include the sensor modules 100 and the connector module 200. The water quality measuring device 10 may acquire water quality data of water desired to be measured from the sensor modules 100, transfer the acquired water quality data to the connector module 200, and transfer the transferred water quality data to the electronic device 20.

For example, the sensor modules 100 may acquire the water quality data through the sensor portions 110, and the water quality data may be calculated or converted by the sensor control portion 140 as data which immediately precedes water quality information such as a value, a graph, an image, a sound, etc.

The water quality data calculated or converted by the sensor control portion 140 may be transferred to the connector module 200 by the sensor-proximate communication portion 120 and the body-proximate communication portion 220, and the transferred water quality data may be transferred to the electronic device 20 by the electronic device communication portion 210.

As described above, the sensor portions 110 may be components which acquire water quality data for measuring hydrogen ions, dissolved oxygen, electric conductivity, BOD, floating particles, total nitrogen, total phosphorus, chloride ions, etc., according to purposes thereof, and the water quality data obtained through the respective sensor portions 110 may be calculated or converted by the sensor control portion 140 as data which immediately precedes water quality information such as a value, a graph, an image, a sound, etc.

Therefore, the electronic device 20 does not require control portions which vary according to purposes thereof and are capable of comprehensively processing the water quality data. As long as the data which immediately precedes the water quality information transferred from the connector module 200 is acquired, the electronic device 20 may display the water quality information to a user.

As a result, a user need not have individual water quality measuring devices 10 for measuring hydrogen ions, dissolved oxygen, electric conductivity, BOD, floating particles, total nitrogen, total phosphorus, chloride ions, etc. according to water quality measurement purposes, and may have only the single connector module 200 and the plurality of sensor modules 100 according to water quality measurement purposes. Therefore, utilization is high.

Alternatively, the body control portion 240 instead of the sensor control portion 140 may comprehensively process the different pieces of water quality data according to purpose.

In other words, the sensor modules 100 may transfer the acquired water quality data to the connector module 200, and the body control portion 240 of the connector module 200 receiving the water quality data from the sensor modules 100 may calculate or convert the water quality data as data immediately preceding the water quality information and may transfer the water quality data to the electronic device 20.

Likewise, the electronic device 20 does not require control portions which vary according to purposes thereof and are capable of comprehensively processing the water quality data. As long as the data which immediately precedes the water quality information transferred from the connector module 200 is acquired, the electronic device 20 may display the water quality information to a user.

As a result, when a user connects the sensor modules 100 for measuring hydrogen ions, dissolved oxygen, electric conductivity, BOD, floating particles, total nitrogen, total phosphorus, chloride ions, etc. to the connector module 200, any one of the sensor control portion 140 and the body control portion 240 calculates or converts data as the water quality data immediately preceding the water quality information. Therefore, the user can obtain the water quality information without additionally manipulating the electronic device 20.

Alternatively, the electronic device 20 instead of the sensor control portion 140 and the body control portion 240 may acquire the water quality information by calculating and processing the water quality data acquired from the sensor modules 100.

In other words, the water quality data acquired from the sensor modules 100 may simply pass through the connector module 200 and may be input to the electronic device 20, and the electronic device 20 may calculate the water quality information from the water quality data.

For example, the connector module 200 and/or the electronic device 20 may determine whether the sensor modules 100 are coupled to the connector module 200. For example, when a case in which the sensor modules 100 are connected to the connector module 200 is compared with a case in which the sensor modules 100 are not connected to the connector module 200, the connector module 200 and/or the electronic device 20 may determine whether the sensor modules 100 are coupled to the connector module 200 based on whether power is wirelessly supplied to the sensor modules 100 or the magnitude of power, whether communication is performed between the sensor modules 100 and the connector module 200 or the intensity of communication, or a change in the distance and the like between the sensor modules 100 and the connector module 200 and/or varying pieces of information.

For example, the sensor modules 100, the connector module 200, and/or the electronic device 20 may be powered on/off according to whether the sensor modules 100 are connected to the connector module 200.

As an example, when the sensor modules 100 are not connected to the connector module 200, the sensor modules 100, the connector module 200, and/or the electronic device 20 may be powered off, and when the sensor modules 100 are connected to the connector module 200, the sensor modules 100, the connector module 200, and/or the electronic device 20 may be powered on.

For example, when the sensor modules 100 are connected to the connector module 200, the connector module 200 may supply power to the sensor modules 100 or communicate with the sensor modules 100, and the sensor modules 100 may be powered on and may acquire the water quality data.

Also, for example, the connector module 200 and/or the electronic device 20 may determine which one of the plurality of sensor modules 100 for measuring hydrogen ions, dissolved oxygen, electric conductivity, BOD, floating particles, total nitrogen, total phosphorus, chloride ions, etc. according to water quality measurement purposes has been connected to the connector module 200.

In other words, as an example, the connector module 200 and/or the electronic device 20 may determine whether the sensor module 100 for acquiring hydrogen ions has been connected to the connector module 200, whether the sensor module 100 for acquiring dissolved oxygen has been connected to the connector module 200, and the like, and may calculate the water quality information on the basis of the water quality data according to water quality measurement purposes.

As an example, unique codes may be given to the sensor module 100 for acquiring hydrogen ions and the sensor module 100 for acquiring dissolved oxygen, and the connector module 200 and/or the electronic device 20 may determine for which one of the purposes a sensor module 100 has been coupled to the connector module 200 by recognizing a unique code of the sensor module 100.

Further, for example, the connector module 200 and/or the electronic device 20 may determine whether the sensor modules 100 are connected to the connector module 200 normally.

As an example, when a case in which the sensor modules 100 are connected to the connector module 200 abnormally is compared with a case in which the sensor modules 100 are connected to the connector module 200 normally, the connector module 200 and/or the electronic device 20 may determine whether the sensor modules 100 are coupled to the connector module 200 normally based on whether power is wirelessly supplied to the sensor modules 100 or the magnitude of power, whether communication is performed between the sensor modules 100 and the connector module 200 or the intensity of communication, or a change in the distance and the like between the sensor modules 100 and the connector module 200 and/or varying pieces information.

Although a configuration and characteristics of the present invention have been described above according to embodiments of the present invention, the present invention is not limited thereto. It is apparent to those of ordinary skill in the art that the present invention may be modified and altered in various ways without departing from the spirit and scope of the present invention. Therefore, such modifications and alterations fall within the scope of appended claims.

1. A sensor module which is connected to a connector module to make an electronic device display water quality information and acquires water quality data for calculating the water quality information, the connector module including an electronic device communication portion for transferring the water quality data for calculating the water quality information to the electronic device, a body-proximate communication portion for wirelessly communicating with the sensor module, and a wireless power transmitting portion for wirelessly supplying power to the sensor module, and the sensor module being detachably connected to the connector module and being connected to an arbitrary connector module, the sensor module comprising:
a sensor portion configured to acquire the water quality data for calculating the water quality information;
a sensor-proximate communication portion configured to wirelessly transmit the water quality data acquired by the sensor portion to the body-proximate communication portion;
a wireless power receiving portion configured to wirelessly receive the power from the wireless power transmitting portion; and
a sensor body portion in which the sensor-proximate communication portion and the wireless power receiving portion are installed,
wherein the sensor body portion is connected to the connector module so that the sensor-proximate communication portion and the wireless power receiving portion are fixed at predetermined positions in the connector module, and
when the sensor body portion and the connector module are connected, the sensor-proximate communication portion and the wireless power receiving portion are positioned to be spaced apart from the body-proximate communication portion and the wireless power transmitting portion so that an abnormal overcurrent is prevented from flowing to at least one of the body-proximate communication portion and the wireless power transmitting portion.

2. The sensor module of claim 1, wherein the sensor body portion include:
a body-connected body portion configured to provide a region to which the connector module is connected;
a sensor-connected body portion configured to provide a region to which the sensor portion is connected; and
a sensor-disposed body portion configured to make the sensor-proximate communication portion and the wireless power receiving portion be positioned at the predetermined positions spaced apart from the body-proximate communication portion and the wireless power transmitting portion.

3. The sensor module of claim 2, wherein the sensor-disposed body portion and the sensor-connected body portion surround the sensor-proximate communication portion and the wireless power receiving portion so that external moisture is prevented from entering into the sensor-proximate communication portion and the wireless power receiving portion.

4. The sensor module of claim 2, wherein the sensor-disposed body portion is attached to or detached from the sensor-connected body portion.

5. The sensor module of claim 2, wherein the sensor-proximate communication portion and the wireless power receiving portion are positioned in the sensor-disposed body portion to be spaced apart from each other in a lengthwise direction of the sensor portion.

6. The sensor module of claim 5, wherein the sensor-disposed body portion makes the sensor-proximate communication portion and the wireless power receiving portion be disposed at the predetermined positions so that the body-proximate communication portion and the wireless power transmitting portion of the connector module are positioned to be spaced apart from the sensor-proximate communication portion and the wireless power receiving portion in a direction perpendicular to the lengthwise direction when the sensor module is connected to the connector module by the body-connected body portion.

7. A connector module which receives water quality data for calculating water quality information from the sensor module of claim 1 including the sensor portion, the sensor-proximate communication portion, and the wireless power receiving portion and transfers the water quality information to an electronic device to display the water quality information on the electronic device, the connector module comprising:
a body-proximate communication portion configured to wirelessly receive the water quality data from the sensor-proximate communication portion of the sensor module;
a wireless power transmitting portion configured to wirelessly supply power to the wireless power receiving portion of the sensor module;
an electronic device communication portion configured to transfer the water quality data to the electronic device; and
a main body portion in which the sensor-proximate communication portion and the wireless power receiving portion are installed,
wherein the main body portion is connected to the sensor module and thereby makes the body-proximate communication portion and the wireless power transmitting portion be fixed at predetermined positions in the sensor module, and
when the main body portion and the sensor module are connected, the body-proximate communication portion and the wireless power transmitting portion are positioned to be spaced apart from the sensor-proximate communication portion and the wireless power receiving portion so that an abnormal overcurrent is prevented from flowing to at least one of the sensor-proximate communication portion and the wireless power receiving portion.

8. The connector module of claim 7, wherein the main body portion includes:
a device-connected body portion configured to be connected to the sensor module; and
a body-disposed body portion configured to make the body-proximate communication portion and the wireless power transmitting portion be positioned at the predetermined positions spaced apart from the sensor-proximate communication portion and the wireless power receiving portion.

9. The connector module of claim 8, wherein the main body portion further includes a communication-connecting body portion configured to provide a region to which the electronic device communication portion is connected, and
the body-disposed body portion and the communication-connecting body portion surround the body-proximate communication portion and the wireless power transmitting portion so that external moisture is prevented from entering into the body-proximate communication portion and the wireless power transmitting portion.

10. The connector module of claim 9, wherein the body-disposed body portion is attached to or detached from the communication-connecting body portion.

11. The connector module of claim 8, wherein the body-proximate communication portion and the wireless power transmitting portion are positioned in the body-disposed body portion to be spaced apart from each other in a lengthwise direction of the sensor portion of claim 1.

12. The connector module of claim 11, wherein the body-disposed body portion makes the body-proximate communication portion and the wireless power transmitting portion be disposed at the predetermined positions so that the sensor-proximate communication portion and the wireless power receiving portion of the sensor module are positioned to be spaced a predetermined distance from the body-proximate communication portion and the wireless power transmitting portion in a direction perpendicular to the lengthwise direction when the connector module is connected to the sensor module by the device-connected body portion.

13. The connector module of claim 8, wherein the device-connected body portion includes:
a first device-connected body portion configured to be connected to the body-disposed body portion; and
a second device-connected body portion configured to connect the first body-connected body portion and the sensor module.

14. The connector module of claim 13, wherein the first device-connected body portion surrounds the sensor-disposed body portion of claim 2 and the body-disposed body portion when the connector module is connected to the sensor module.

15. The connector module of claim 8, wherein the main body portion further includes a communication-connecting body portion configured to provide a region to which the electronic device communication portion is connected, and
the body-disposed body portion includes:
a first body-disposed body portion configured to make the body-proximate communication portion and the wireless power transmitting portion be disposed at the predetermined positions;
a second body-disposed body portion configured to provide a region to which the communication-connecting body portion is connected;
a third body-disposed body portion configured to provide a region to which the device-connected body portion is connected; and
a fourth body-disposed body portion configured to define a boundary between the second body-disposed body portion and the third body-disposed body portion.

16. A water quality measuring device comprising:
the sensor module of claim 1; and
the connector module of claim 7.
